(19) United States
(12) Patent Application Publication
(10) Pub. No.: US 2010/0152789 A1
(43) Pub. Date: Jun. 17, 2010

(54) Skin-Bone Clamp

(76) Inventor: Alberto A. Fernandez DELL'OCA, Montevideo (UY)

Correspondence Address: Fay Kaplun & Marcin, LLP, 150 Broadway, suite 702, New York, NY, 10038, US

(21) Appl. No.: 12/635,091

(22) Filed: Dec. 10, 2009

Related U.S. Application Data
(60) Provisional application No. 61/121,199, filed on Dec. 10, 2008.

(51) Int. Cl. A61B 17/84

(52) U.S. Cl. 606/324

(57) ABSTRACT

A bone clamping device, comprises first and second arms pivotally connected to one another, the first arm including a distal end configured to directly contact a portion of bone to be clamped and the second arm including a distal end comprising a substantially spherical tip and an insert configured for attachment to the spherical tip of the second arm, the insert comprising a first face having an opening for receiving the spherical distal tip of the second arm and a second face opposite the first face, the second face having a contour selected to match a contour of a portion of skin against which the insert is to be positioned when in an operative configuration.

### PRIORITY CLAIM

The present application claims priority to U.S. Provisional Application Ser. No. 61/121,199, entitled “Skin-Bone Clamp” filed on Dec. 10, 2008. The entire disclosure of the above-identified application is incorporated herewith by reference.

### BACKGROUND

Clamps are commonly used in bone fixation procedures to correct the alignment of bone fragments and to hold the bone fragments in the corrected alignment until a permanent fixation device can be applied to the bone. Typical bone clamps require the use of a surgical approach to permit gripping ends of the bone clamps to come into direct contact with the target portions of the bone to apply a clamping force thereto. These bone clamps are inserted through a large incision formed through the skin adjacent to the fracture site or through multiple openings formed at a plurality of predetermined positions adjacent to the target region. Such bone clamps generally comprise towel-clip gripping end portions having sharpened opposing points to permit a non-slip grasping of the bone.

### SUMMARY OF THE INVENTION

The present invention is directed to a bone clamping device comprising first and second anus pivotally connected to one another, the first arm including a distal end configured to directly contact a portion of bone to be clamped and the second arm including a distal end comprising a substantially spherical tip and an insert configured for attachment to the spherical tip of the second arm, the insert comprising a first face having an opening for receiving the spherical distal tip of the second arm and a second face opposite the first face, the second face having a contour selected to match a contour of a portion of skin against which the insert is to be positioned when in an operative configuration.

The present invention is further directed to a method for clamping a bone, comprising positioning a distal end of a first arm of a bone clamp against a surface a first target portion of bone via a minimally invasive incision, the bone clamp comprising first and second arms pivotally connected to one another and positioning a distal end of the second arm against a portion of skin adjacent to a second target portion of bone, the distal end of the second arm comprising a foot contoured to match a contour of the portion of skin against which it is to be positioned in combination with drawing the distal ends of the first and second arms toward one another to apply a compressive force between the first and second target portions of bone.

### BRIEF DESCRIPTION OF THE DRAWINGS

FIG. 1 shows a perspective view of a system according to a first exemplary embodiment of the present invention;

FIG. 2 shows a zoomed view of the system of FIG. 1 in an operative configuration against a bone;

FIG. 3 shows a zoomed view of a distal end of the arm of FIG. 1;

FIG. 4 shows another perspective view of FIG. 3;

FIG. 5 shows a zoomed view of a distal end of an arm according to a first alternate embodiment of the present invention; and

FIG. 6 shows another perspective view of FIG. 5.

### DETAILED DESCRIPTION

The present invention may be further understood with reference to the following description and the appended drawings, wherein like elements are referred to with the same reference numerals. The exemplary embodiments of the present invention relate to a system and method for the reduction of fractured, shattered or otherwise damaged bones by a bone clamp. The exemplary bone clamp of the present invention may be used for the fixation of any fracture and comprises a pair of forceps arms having distal ends configured to aid in reduction of the bone. A first one of the arms comprises a sharpened distal end configured to directly contact the bone through, for example, a minimally invasive incision. A second one of the arms comprises a rounded or otherwise blunted distal end configured to indirectly engage an opposing face of the bone (e.g., by being pressed against the skin surrounding the bone) and thus finds utility in minimally invasive procedures. As will be described in greater detail hereinafter, the second distal end may be configured to engage a portion of skin located adjacent to a portion of the bone opposing the fracture site to prevent the need for a large incision or for multiple incisions.

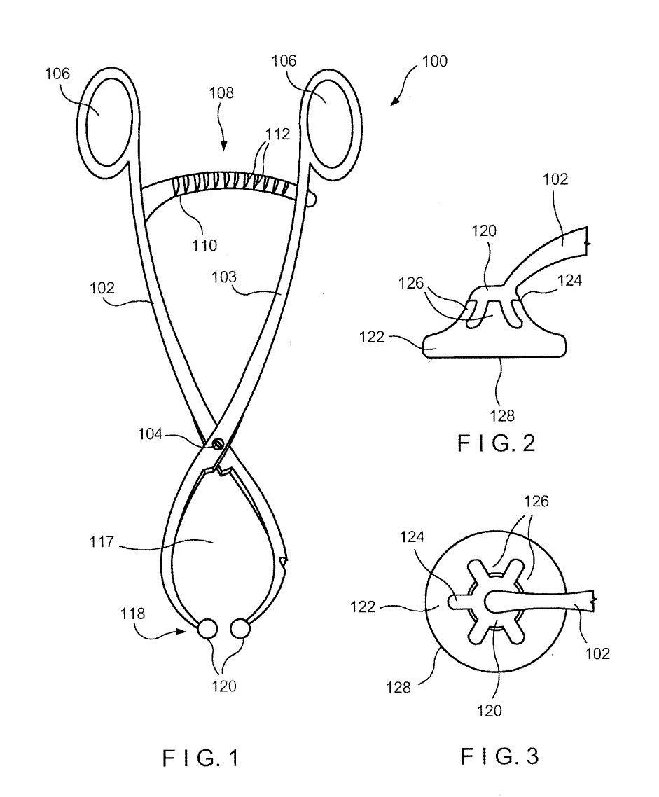

As shown in FIGS. 1-3, a clamping device 100 according to a first exemplary embodiment of the present invention comprises first and second arms 102, 103 joined together at a pivot point 104. Proximal ends of each of the arms 102, 103 comprise finger loops 106 configured to permit gripping and actuation of the arms 102, 103, as those skilled in the art will understand. The arms 102, 103 also comprise a ratcheting locking mechanism 108 at their proximal ends adjacent to the finger loops 106. Specifically, a tabbed protrusion 110 extending from the first arm 102 toward the second arm 103 includes a plurality of raised tabs 112 formed on a first wall thereof. The tabbed protrusion 110 extends along an arc substantially centered at the pivot point 104 and has a length selected so that, as distal ends 118 are drawn together, the second arms 103 comes into contact with the tabs 112 locking the arms 102, 103 in a ratchet-like manner. Specifically, recesses are defined between the tabs 112 configured to seat the second arm 103 while a face of each of the tabs facing 112 toward the second arm 103 is angled to permit the second arm 103 to slide thereover into the following recess while a face of each arm facing away from the second arm 103 extends straight out from the tabbed protrusion to lock the second arm 103 and prevent it from slipping thereover away from the first arm 102. This temporarily locks a position of the first and second aims 102, 103 relative to one another, as those skilled in the art will understand.

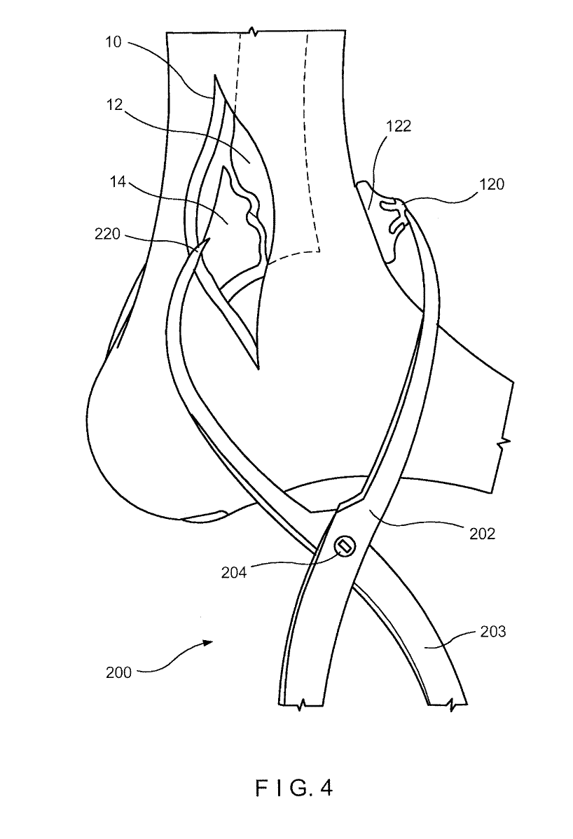

Portions of each of the first and second arms 102, 103 located distally of the pivot point 104 initially curve outward away from one another and then curve back toward one another so that, when the distal ends 118 thereof are brought into contact with one another, a space 117 is defined between these distal portions of the first and second arms 102, 103. The ends 118 comprise substantially spherical balled tips 120. In an exemplary embodiment, the arms 102, 103 and tips 120 are both formed of a suitable biocompatible material (e.g., stainless steel) as would be understood by those skilled in the art. As those skilled in the art will understand, the balled tips 120 permit the application of a constrictive force to soft tissue adjacent to a target bone without causing trauma thereto. That is, whereas conventional bone clamps employ dual sharpened distal tips to aid in fracture reduction, a large incision or multiple incisions are required to permit these tips to come into direct contact with the bone. In contrast, the balled tips 120 of this embodiment of the invention permit the clamping of bone with only a minimally invasive incision as will be described below. The balled tips 120 are shaped and sized to receive plastic inserts 122 thereover, as shown in FIGS. 2 and 3. Specifically, the plastic insert 122 has a substantially spherical opening 124 formed into a first wall thereof sized and shaped to compressively receive the balled tip 120 therein. The opening 124 is bordered by a plurality of fingers 126 configured to flex away from the insert 122 by a distance sufficient to permit slidable insertion of the balled tip 120 into the opening 124 and which are biased to return to their original position so that they grip the balled tip 120 and retain it within the opening 124. A side of the insert 122 opposite the opening 124 is formed as a substantially planar face 128 configured to rest against a target portion of soft tissue in an operative configuration, as will be described in greater detail later on. As those skilled in the art will understand, the substantially spherical shape of the opening 124 permits the balled tip 120 to rotate when positioned therewithin permitting pivotal movement of the insert 122 relative to the arm 102. The pivotal movement allows the insert 122 to adjust to the contour of a portion of skin with which the planar face 128 is in contact increasing an area of contact therewith to prevent slipping. It is noted that although the insert 122 is shown with a circular profile, the insert 122 may be formed with any shape and size without deviating from the spirit and scope of the present invention so long as the shape is selected to conform to the requirements of the procedure being performed. The exemplary system according to the present invention permits the use of the clamping device in minimally invasive bone fixation procedures where, for example, only one incision is to be made adjacent a bone fracture site, as will be described in greater detail with respect to the system 200 below. That is, in any given procedure the user may select one of the ends 120 for insertion through a minimally invasive incision to directly contact the bone. The other end 120 will be inserted into an insert 122 to enhance the stability of the clamping device 100 as it applies compressive force to the bone by being pressed against the skin adjacent to the bone.

FIG. 4 depicts a clamping device 200 according to a first alternate embodiment of the present invention. The clamping device 200 is formed substantially similarly to the clamping device 100 of FIG. 1 except for the shapes of the distal ends of the arms. Specifically, the clamping device 200 comprises first and second arms 202, 203 connected to one another at a pivot point 204. The first arm 202 is formed substantially similarly to the first arm 102 of FIG. 1 and comprises the insert 122 connected to the balled tip 120. A distal end 218 of the second arm 204 gradually reduces in diameter from the pivot point 204 to a sharpened distal tip 220 configured to be placed in direct contact with the bone via a minimally invasive incision as those skilled in the art will understand to aid in gripping the bone. Thus, while this clamping device 200 has an enhanced grip against the surface of the bone, the device 200 can not be configured as desired by the surgeon in the same manner as the device 100—i.e., the insert 122 can be placed only on the balled tip 120 of the first arm 202.

In accordance with an exemplary method according to the present invention, a minimally invasive incision 10 is formed through the skin adjacent a site at which a fragment 14 is separated from a tibia 12. It is noted that although the method is described with respect to the fixation of a tibial fracture, the exemplary clamping devices 100, 200 may be used for the fixation of any bone in the body. The clamping device 200 is used to reduce the bone in preparation for permanent fixation via, for example, a bone plate (not shown). The distal tip 220 of the second arm 203 is inserted through the incision 10 into contact with the second bone fragment 14 while the insert 122 on the distal end 118 of the first arm 202 is positioned against the skin over an anterior portion of the ankle at a location substantially opposing the location of the fragment 14. Specifically, the insert 122 of the first arm 202 is separated from the tip 220 of the second arm 203 in the direction of desired movement of the fragment 14 so that, as the first and second arms 202, 203, respectively, are drawn toward one another, the fragment 14 is moved against the tibia 12 to reduce the fracture. In the embodiment of FIG. 4, the fragment 14 must be moved in an anterior direction to correct alignment thereof with the tibia 12. As the planar wall 128 of the insert 122 is placed over the skin, the insert 122 pivots relative to the balled tip 120 until the planar wall 128 is firmly seated in a contacting configuration against the skin. As noted earlier, the pivotal movement of the insert 122 relative to the balled tip 120 permits the insert 122 to conform the curvature of the skin so that a compressive force applied thereby is evenly distributed over the entire planar wall 128, thus minimizing and/or preventing trauma to the tissue as a result of the compressive force. The first and second arms 202, 203 are then manipulated until the fragment 14 is brought into a desired alignment against the tibia 12. As the distal ends of the arms 202, 203 are drawn together, the ratcheting mechanism 108 prevents the arms 202, 203 from being inadvertently drawn apart and allows the user to release the device 200 while maintaining a desired compressive force on the fragment 14 and the tibia 12. When the fracture has been stabilized (e.g., through the application of a bone plate), the user disengages the tabs 112 from the second arm 203 by applying a force a plane substantially perpendicular to a plane housing the arms 202, 203 releasing the arms 202, 203 to pivot relative to one another for removal of the device 200.

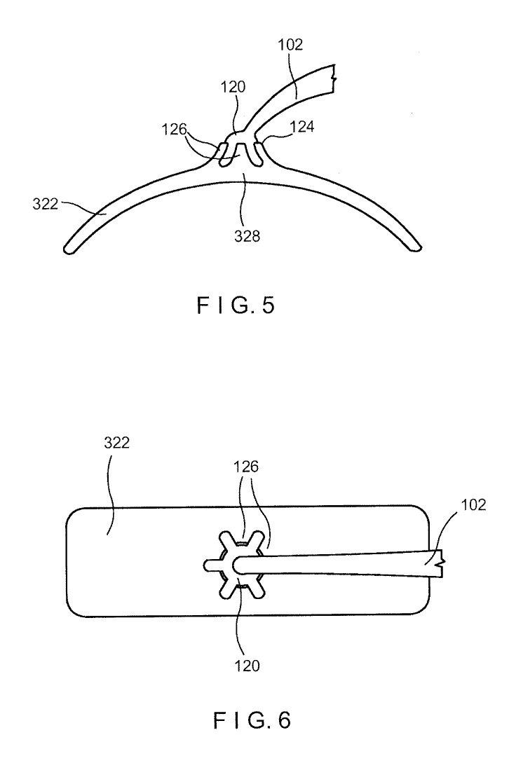

As shown in FIGS. 5-6, an insert 322 according to another embodiment of the present invention is substantially similar to the insert 122 of FIGS. 2-4 with the exception of a shape thereof. Specifically, the insert 322 also comprises a substantially spherical opening 124 with retractable fingers 126 configured to compressively engage the balled tip 120 inserted therein. However, instead of a planar face 128, the insert 322 comprises a convex face 328 having a curvature configured to match a curvature of a portion of the anatomy which the insert 322 is to contact when in an operative configuration. It is further noted that although the insert 322 is shown with a rectangular outer profile, any shape, dimensions and curvature may be applied thereto to conform to the requirements of a procedure being performed.

Furthermore, it is noted that although the bone clamping devices 100, 200 shown are substantially planar, the arms 102, 103, 202, 203 may be angled as needed for ergonomic handling and/or to conform to the anatomy of the region of the body being treated.

It will be apparent to those skilled in the art that various modifications and variations can be made in the structure and the methodology of the present invention, without departing from the spirit or the scope of the invention. Thus, it is intended that the present invention cover the modifications and variations of this invention provided that they come within the scope of the appended claims and their equivalents.

What is claimed is:

1. A bone clamping device, comprising:
first and second arms pivotally connected to one another, the first arm including a distal end configured to directly contact a portion of bone to be clamped and the second arm including a distal end comprising a substantially spherical tip; and
an insert configured for attachment to the spherical tip of the second arm, the insert comprising a first face having an opening for receiving the spherical distal tip of the second arm and a second face opposite the first face, the second face having a contour selected to match a contour of a portion of skin against which the insert is to be positioned when in an operative configuration.

2. The device of claim 1, wherein the opening in the insert being substantially spherical so that insertion of the spherical tip of the second arm pivotally connects the insert to the second arm.

3. The device of claim 1, wherein the first arm includes a distal end comprising a substantially spherical tip.

4. The device of claim 3, wherein the spherical tip of the first arm is substantially the same size as the spherical tip of the second arm so that a user may attach the insert to either of the first and second arms.

5. The device of claim 1, wherein the distal end of the first arm forms a sharpened bone engaging tip.

6. The device of claim 1, further comprising a plurality of fingers extending around a border of the opening, the fingers being biased to compressively engage the spherical distal tip of the second arm.

7. The device of claim 1, further comprising a ratchet mechanism temporarily locking a position of the first and second arms relative to one another until released.

8. A method for clamping a bone, comprising:
positioning a distal end of a first arm of a bone clamp against a surface a first target portion of bone via a minimally invasive incision, the bone clamp comprising first and second arms pivotally connected to one another;
positioning a distal end of the second arm against a portion of skin adjacent to a second target portion of bone, the distal end of the second arm comprising a foot contoured to match a contour of the portion of skin against which it is to be positioned;
drawing the distal ends of the first and second arms toward one another to apply a compressive force between the first and second target portions of bone.

9. The method of claim 8, wherein the foot is pivotally connected to the second arm.

10. The method of claim 9, wherein the foot includes an opening configured to pivotally receive therein a spherical tip of the second arm.

11. The method of claim 10, wherein the first arm includes a spherical tip substantially similar to the spherical tip of the second arm so that the foot may be attached to either of the first and second arms.

12. The method of claim 8, wherein the distal end of the first arm includes a sharpened distal tip, wherein the step of positioning the distal end of the first arm against a surface of the first target portion of bone includes engaging the first target portion of the bone with the sharpened distal tip of the first arm.
